(19) United States
(12) Patent Application Publication
(10) Pub. No.: US 2024/0287027 A1
(43) Pub. Date: Aug. 29, 2024

(54) PREPARATION METHOD FOR COMPOUND FASUDIL HYDROCHLORIDE

(71) Applicant: EAST CHINA UNIVERSITY OF SCIENCE AND TECHNOLOGY, Shanghai (CN)

(72) Inventors: Jianhong ZHAO, Shanghai (CN); Dingding WANG, Shanghai (CN); Wulin YANG, Shanghai (CN); Jinming NIU, Shanghai (CN); Jun ZHU, Shanghai (CN); Weiting WU, Shanghai (CN); Jie YU, Shanghai (CN); Zhen SUN, Shanghai (CN); Xiaoli XU, Shanghai (CN)

(21) Appl. No.: 18/570,511

(22) PCT Filed: May 26, 2022
(86) PCT No.: PCT/CN2022/095330

§ 371 Date: Dec. 14, 2023

(30) Foreign Application Priority Data
Jun. 15, 2021 (CN) 202110661695.8

(51) Int. Cl. C07D 401/12

(52) U.S. Cl. CPC C07D 401/12

(57) ABSTRACT

An efficient and economical method for synthesizing Fasudil hydrochloride includes the synthesis route starts with inexpensive and readily available ethylenediamine as the starting material, obtains intermediate tert butyl-(N-(2-aminoethyl)-5-isoquinoline sulfonamide) carbamate (5) through sulfonylation and Boc protection, and then obtains Fasudil hydrochloride through a stacking process, which includes four steps: nucleophilic substitution, deprotection, cyclization, and salt formation. The total yield of Fasudil hydrochloride (1) is 67.1%, with a purity of up to 99.94%. Compared with traditional processes, the route avoids the use of expensive homopiperazine and its derivatives as synthetic intermediates. The advantages of the process include cheap and easy to obtain raw materials, simple operation, low cost, environmental friendliness, and suitability for industrial production.

### CROSS REFERENCING OF RELATED APPLICATIONS

This application claims the priority of the Chinese invention patent application with application Ser. No. 20/211,0661695.8 filed on Jun. 15, 2021, and its content is incorporated into this application by reference.

### FIELD OF THE INVENTION

The present invention relates to the field of drug preparation, in particular to a new process for the preparation of Active Pharmaceutical Ingredient Fasudil hydrochloride.

### BACKGROUND OF THE INVENTION

Fasudil Hydrochloride, chemical name hexahydro-1-(5-sulfonylisoquinoline)-1 (H)-1,4-diazazene hydrochloride, has the following structural formula:

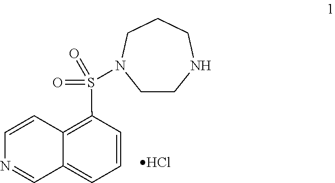 [SMILES: O=S(=O)(c1cccc2cnccc12)N1CCCNCC1 ; Cl]

Fasudil hydrochloride is a novel cardiovascular drug with extensive pharmacological effects. As an RHO kinase inhibitor and a novel intracellular Ca²⁺ antagonist, it has a good vasodilator effect. By increasing the activity of myosin light chain phosphatase (MLCP), it dilates blood vessels, reduces endothelial cell tension, improves brain tissue microcirculation, improves the prognosis of subarachnoid hemorrhage (SAH) patients without producing or exacerbating cerebral hemorrhage, and can also block anti-inflammatory factors, protect nerves from apoptosis, and promote nerve regeneration. This drug was developed by Asahi Kasei Co., Ltd. in Japan in the 1980s, launched in June 1995, and launched in China in 2004. It is mainly used to improve symptoms of ischemic cerebrovascular diseases caused by cerebral vasospasm after subarachnoid hemorrhage. Its clinical application scope will continue to expand, and the market prospects are promising.

At present, there are not many reported preparation methods both domestically and internationally. The existing publicly available preparation processes are as follows:

The preparation route adopted by Asahi Kasei patent U.S. Pat. No. 4,678,783 (original research patent) involves the sulfonation of isoquinoline with fuming sulfuric acid to obtain 5-isoquinoline sulfonic acid. The 5-isoquinoline sulfonic acid reacts with sulfoxide chloride under DMF catalysis to obtain a solid 5-isoquinoline sulfonyl chloride, which is then dissociated with sodium bicarbonate aqueous solution (pH adjusted to around 6.0), and then extracted with dichloromethane to obtain a dichloromethane solution of 5-isoquinoline sulfonyl chloride. Then, it reacts with homopiperazine through acylation condensation to obtain fasudil (purified by silica gel column chromatography), which is then salted with hydrochloric acid to obtain fasudil hydrochloride. The synthesis method of this process is shown in FIG. 1.

Patent CN101863880 relates to the sulfonation step of using chlorosulfonic acid instead of fuming sulfuric acid in traditional processes to prepare 5-isoquinoline sulfonic acid. The subsequent steps are almost the same as patent U.S. Pat. No. 4,678,783, which involves preparing 5-isoquinoline sulfonyl chloride hydrochloride under SOCl₂/DMF conditions, after adjusting the pH value to around 6 with sodium bicarbonate aqueous solution, it is extracted with dichloromethane. The resulting dichloromethane solution is then condensed with homopiperazine.

Patent CN101973982 improves the design of the reaction connecting the homopiperazine group in the above patent method, using a protective group to protect the N atom on the homopiperazine ring, improving the selectivity of acylation reaction and avoiding the formation of dimer. The process route is shown in FIG. 2, and patent CN102120739 synthesizes a homopiperazine derivative with a protective group using 4-piperidone hydrochloride salt solution as the raw material to improve the selectivity of acylation reaction, which is the same as this process principle.

Patent CN102020636 relates to a purification method for Fasudil hydrochloride, which involves treating the dichloromethane solution of Fasudil by acid-base treatment. Specifically, 5-isoquinoline sulfonic acid is first treated under SOCl₂/DMF conditions to prepare 5-isoquinoline sulfonic acid hydrochloride. The pH value is adjusted to neutral with NaHCO₃ aqueous solution, and then extracted with dichloromethane. The dichloromethane solution reacts with homopiperazine to obtain a dichloromethane solution of Fasudil. The pH of the solution was adjusted to 4.5-5.5 with acid solution, and then the aqueous phase was extracted with dichloromethane. The organic phase containing dimer impurities was discarded, and the resulting aqueous phase was adjusted to 9.5-10.5 with alkaline solution. Then, the aqueous phase was extracted with dichloromethane, and the aqueous phase containing homopiperazine impurities was discarded. The solution was purified by silica gel column chromatography and salted with hydrochloric acid to obtain Fasudil hydrochloride. The method of purifying Fasudil's dichloromethane solution through acid-base treatment has also been used in the implementation case of patent U.S. Pat. No. 5,942,505.

Chinese patents CN102002036, CN101812051, CN101962379, CN101092413, and CN101723934 all involve how to purify and refine Fasudil hydrochloride, including methods such as changing column chromatography purification eluent, changing recrystallization solvent, and resin adsorption.

The above reported synthesis routes all involve the use of expensive homopiperazine or its derivatives as synthetic intermediates. The synthesis method of homopiperazine is reported by Reference Wang Daolin, Qian Jianhua, et al. A new synthesis method for homopiperazine [J]. Chemical Reagent, 205. (05): 311-312., using ethylenediamine as the raw material through three steps of sulfonation, cyclization, and desulfonylation, with a yield of 78%. The synthesis route is shown in FIG. 3.

In summary, the process routes for preparing Fasudil hydrochloride all use expensive homopiperazine and its derivatives as synthetic intermediates, resulting in extremely high raw material costs and many process drawbacks: 1) The synthesis process route of homopiperazine is complex, with low atomic economy and high cost; 2) A large amount of three wastes are generated during the synthesis process; 3) The low selectivity of the reaction between 5-isoquinoline sulfonyl chloride and homopiperazine results in a large amount of homopiperazine, further increasing the raw material cost; 4) Multiple reaction steps, long production cycle, and low industrial production efficiency.

### SUMMARY OF THE INVENTION

The purpose of the present invention is to overcome the shortcomings of the prior art and provide a safe, controllable, efficient, and simple method for preparing compound Fasudil hydrochloride.

The present invention provides a method for preparing compound hydrochloric acid fasudil, and the synthesis route is shown in FIG. 4.

The method comprising steps:
(1) dissolving 5-isoquinoline sulfonyl chloride in a first organic solvent, then adding dropwise to a mixed solvent of ethylenediamine, solid base, and the first organic solvent, react at 20-25° C. for 2-5 hours, and then subjected to acid-base treatment to obtain N-(2-aminoethyl)-5-isoquinoline sulfonamide (4);
(2) Mixing the N-(2-aminoethyl)-5-isoquinoline sulfonamide (4) obtained in step (1), base, and a second organic solvent with water, stirring and adding (Boc)₂O solution dropwise, after dropwise addition, reacting for 0.5-2 hours, extracting and separating reaction system after reaction, drying an organic phase and concentrating to obtain tert butyl-(2-(5-isoquinoline sulfonamide)ethyl) carbamate (5);
(3) Dissolving the tert butyl-(2-(5-isoquinoline sulfonamide)ethyl) carbamate (5) obtained in step (2) in a third organic solvent, and undergoing nucleophilic substitution reaction with 1,3-disubstituted propane at 50-55° C. to obtain compound (6);
(4) Deprotecting compound (6) obtained in step (3) under acidic conditions, then cyclizing under alkaline conditions to obtain fasudil, and then salt with hydrochloric acid to obtain fasudil hydrochloride (1).

Preferably, the acid-base treatment is carried out as follows:
Adding hydrochloric acid solution dropwise to reaction solution for adjusting pH to 2-3, and then separating solution, discarding an organic layer;
Dripping an alkaline solution into a water layer for adjusting pH to 7-8, wherein the alkaline solution is sodium bicarbonate, potassium bicarbonate, sodium hydroxide, potassium hydroxide, sodium carbonate or potassium carbonate, preferably sodium hydroxide aqueous solution;
Stirring to precipitate white solid, adding saturated salt water to promote solid precipitation, filtering, and drying filter cake to obtain N-(2-aminoethyl)-5-isoquinoline sulfonamide.

Preferably the first organic solvent is one or more of dichloromethane, dichloroethane, ethylenediamine, triethylamine, diisopropylethylamine, trimethylamine, pyridine, toluene, ethyl acetate, methanol, ethanol, tetrahydrofuran or acetonitrile, preferably dichloromethane; the second organic solvent is one or more of tetrahydrofuran, dioxane, acetonitrile, methanol, or ethanol, preferably tetrahydrofuran;

the third organic solvent is one or more of dichloromethane, 1,2-dichloroethane, tetrahydrofuran, dioxane, acetonitrile, methanol, ethanol, ethyl acetate, isopropyl acetate, methyl isobutyl ketone, toluene, isopropyl acetate, N, N-dimethylformamide, N, N-dimethylacetamide, or dimethyl sulfoxide, preferably tetrahydrofuran.

Preferably in step (1), the solid base is sodium bicarbonate, potassium bicarbonate, sodium hydroxide, potassium hydroxide, sodium carbonate or potassium carbonate, preferably sodium bicarbonate or potassium bicarbonate;

in step (2), the base is sodium carbonate, potassium carbonate, sodium bicarbonate, potassium bicarbonate, sodium hydroxide, potassium hydroxide, or triethylamine, preferably potassium carbonate or sodium carbonate;

in step (3), the substituent is chlorine (CI), bromine (Br), p-toluenesulfonate (TsO), or methanesulfonate; Preferably 1-bromo-3-chloropropane;

In step (4), base used for cyclization under alkaline conditions is NaOH, KOH, Na₂CO₃, NaHCO₃, KHCO₃, or triethylamine, preferably NaOH.

Preferably in step (4), a deprotection condition is a hydrochloric acid/methanol, hydrochloric acid/ethanol, or trifluoroacetic acid/dichloromethane system at a temperature of 50-55° C., preferably a hydrochloric acid/methanol system.

The preparation method of the compound Fasudil hydrochloride of the present invention uses inexpensive and readily available ethylenediamine as the starting material and 1-bromo-3-chloropropane as the most economical cyclization reagent, and synthesize homopiperazine ring in steps, avoiding the use of expensive homopiperazine and its derivatives. The reaction conditions are warm, the operation is simple, the cost is low, the environment is friendly, and it is suitable for industrial production.

### BRIEF DESCRIPTION OF THE DRAWINGS

FIG. 1 shows the process flow diagram of the synthesis method in Japanese Asahi Kasei patent U.S. Pat. No. 4,678,783.

FIG. 2 is a flowchart of the process synthesis method in patent CN101973982.

FIG. 3 shows the process flow diagram of the synthesis method in the literature by Wang Daolin et al.

FIG. 4 is a flowchart of the preparation method for the compound hydrochloric acid fasudil of the present invention.

FIG. 5 shows the HPLC image of Fasudil hydrochloride obtained from Example 4 of the present invention.

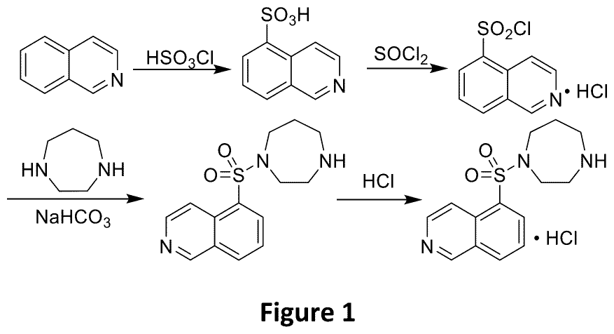

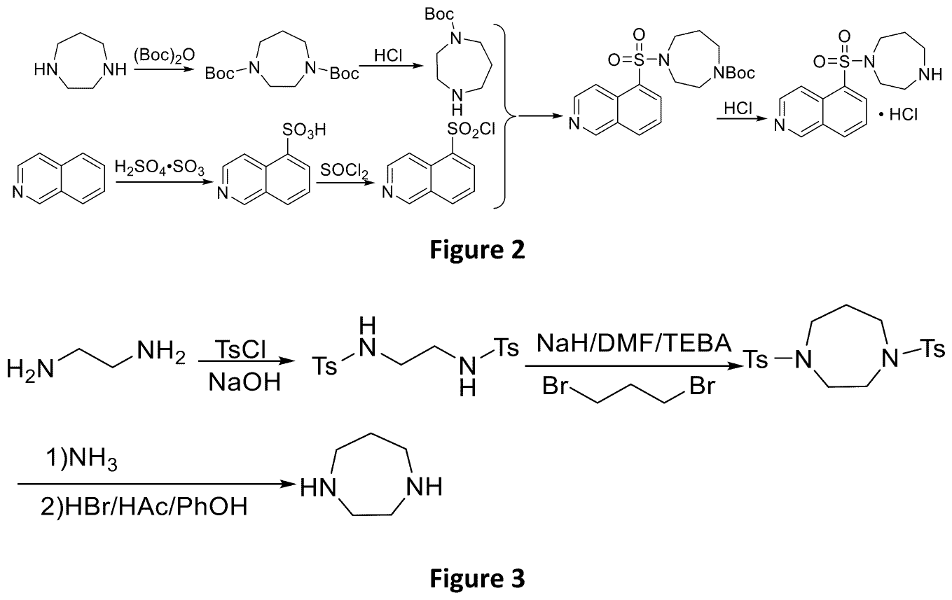

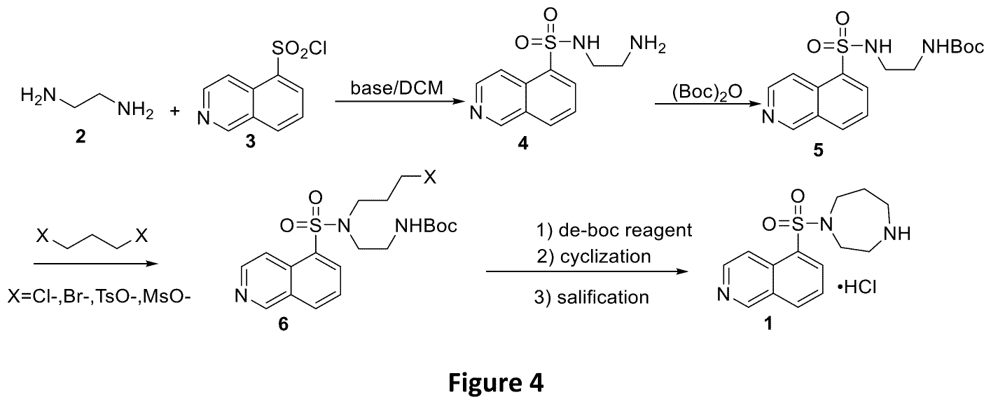

### DETAILED DESCRIPTION OF THE INVENTION

In order to better describe the technical content of the present invention and verify the role of IGFBP2 in early-life stress-induced psychic trauma, further description will be given in conjunction with specific embodiments below.

The present invention relates to an efficient and economical new process for synthesizing Fasudil hydrochloride. The synthesis route starts with inexpensive and readily available ethylenediamine as the starting material, obtains intermediate tert butyl-(N-(2-aminoethyl)-5-isoquinoline sulfonamide) carbamate (5) through sulfonylation and Boc protection, and then obtains Fasudil hydrochloride through a stacking process, which includes four steps: nucleophilic substitution, deprotection, cyclization, and salt formation. The total yield of Fasudil hydrochloride (1) is 67.1%, with a purity of up to 99.94%. Compared with traditional processes, the route avoids the use of expensive homopiperazine and its derivatives as synthetic intermediates. The advantages of the process include cheap and easy to obtain raw materials, simple operation, low cost, environmental friendliness, and suitability for industrial production.

Specifically, the present invention provides a method for preparing a compound fasudil hydrochloride, comprising:
(1) dissolving 5-isoquinoline sulfonyl chloride in a first organic solvent, then adding dropwise to a mixed solvent of ethylenediamine, solid base, and the first organic solvent, react at 20-25° C. for 2-5 hours, and then subjected to acid-base treatment to obtain N-(2-aminoethyl)-5-isoquinoline sulfonamide (4), wherein process the reaction solution as follows:
   (a) Adding hydrochloric acid solution dropwise to reaction solution for adjusting pH to 2-3, and then separating solution, discarding an organic layer;
   (b) Dripping a alkaline solution into a water layer for adjusting pH to 7-8;
   (c) Stirring to precipitate white solid, adding saturated salt water to promote solid precipitation, filtering, and drying filter cake to obtain N-(2-aminoethyl)-5-isoquinoline sulfonamide (4).
(2) Mixing the N-(2-aminoethyl)-5-isoquinoline sulfonamide obtained in step (1), base, and a second organic solvent with water, stirring and adding (Boc)₂O solution dropwise, after dropwise addition, reacting for 0.5-2 hours, extracting and separating reaction system after reaction, drying an organic phase and concentrating to obtain tert butyl-(2-(5-isoquinoline sulfonamide)ethyl) carbamate (5);
(3) Dissolving the tert butyl-(2-(5-isoquinoline sulfonamide)ethyl) carbamate (5) obtained in step (2) in a third organic solvent, and undergoing reaction with 1,3-disubstituted propane (preferably 1-bromo-3-chloropropane) at 50-55° C. to obtain compound (6);
(4) Deprotecting compound (6) obtained in step (3) under acidic conditions, then cyclizing under alkaline conditions to obtain fasudil, and then salt with hydrochloric acid to obtain fasudil hydrochloride (1).

### Example 1 Preparation of N-(2-aminoethyl)-5-isoquinoline sulfonamide hydrochloride (4)

Dissolve 5-isoquinoline sulfonyl chloride (86.5 g) in 500 ml of dichloromethane, then slowly drop it into a mixture of ethylenediamine (2) (99%, 68.6 g), solid NaHCO₃(99%, 63.68 g), and 100 ml of dichloromethane. After dripping, react for 2 hours; Add concentrated HCl dropwise to the reaction solution, adjust the pH≈2, obtain a transparent clear solution, separate the solution, and discard the organic layer; Drip 40% NaOH solution into the water layer to adjust the pH to ≈8, stir and precipitate at 0-5° C. for 2 hours, filter, wash the filter cake with ethanol (200 mL*2), dry at 45° C. for 2 hours, and obtain 107.1 g of white powdery solid (compound 4). mp: 249.9-250.8° C. Literature (253-254° C.). MS-ESI: m/z 252.1, [M+H]⁺ (100%); 1H NMR (400 MHZ, D20) δ (ppm): 9.04 (s, 1H), 8.41 (d, J=6.2 Hz, 1H), 8.27 (d, J=6.2 Hz, 1H), 8.20 (dd, J1=7.4, J2=1.2 Hz, 1H), 8.04 (d, J=8.3, 1H), 7.59 (t, J=7.8 Hz, 1H), 2.92 (t, J=5.8 Hz, 2H), 2.68 (t, J=6.2, 2H).

### Example 2 Preparation of tert butyl-(N-(2-aminoethyl)-5-isoquinoline sulfonamide) carbamate (5)

At room temperature, place the N-(2-aminoethyl)-5-isoquinoline sulfonamide (4) (107.1 g) obtained in Example 1 into a 1000 mL three necked bottle, add 450 ml of tetrahydrofuran and 150 ml of water, stir to obtain a white turbid solution, add anhydrous potassium carbonate (99%, 92.2 g), stir for 30 minutes, then add (Boc)₂O tetrahydrofuran solution dropwise (99%, 72.8 g, 100 ml of tetrahydrofuran), monitor the reaction process by TLC, and stir for 30 minutes after complete conversion of the raw materials. Add 100 ml of water, 200 ml of methyl tert ether, stir and extract, anhydrous magnesium sulfate was added to the organic layer, stir and dry for 2 hours, after filtration, the filtrate was concentrated under reduced pressure to remove the solvent, resulting in a white solid, and then add ethyl acetate for pulping to obtain 113.7 g of white solid (5), mp: 146.8-147.9° C. MS-ESI: m/z 352.1, [M+H]⁺; 1H NMR (400 MHZ, CDCl₃) δ (ppm): 9.33 (s, 1H), 8.61 (d, J=5.9 Hz, 1H), 8.41-8.39 (m, 2H), 8.18 (d, J=8.2 Hz), 7.67 (t, J=7.9 Hz, 1H), 6.39 (t, J=5.8 Hz, 1H), 5.02 (s, 1H), 3.19 (q, J=5.8 Hz, 2H), 3.04 (q, J=5.7 Hz, 2H), 1.35 (s, 9H).

### Example 3 Preparation of tert butyl-(2-(N-(3-chloropropane) 5-isoquinoline sulfonamide)ethyl) carbamate (6)

Place solid 5 (113.7 g) obtained from Example 2 into a 1000 ml three necked bottle, add tetrabutylammonium bromide (99%, 5.3 g, 16.3 mmol, 0.05 eq), sodium hydroxide (96%, 27.1 g), add 400 ml of tetrahydrofuran and 30 ml of water, and stir to obtain a colorless clear solution; Take another 1000 mL three necked bottle, add 1-bromo-3-chloropropane (99%, 77.5 g) and 200 mL tetrahydrofuran, raise the temperature to 50-55° C., add the colorless clear liquid dropwise, react for 4-5 hours after dropwise addition, and drop the temperature of the raw material to room temperature after complete conversion; Add 100 ml of water, stir with 100 mL of tetrahydrofuran, separate the solution, and discard the water layer; the concentrated solution of compound (6) obtained by vacuum concentration of the organic layer is directly put into the next reaction without purification.

### Example 4 Preparation of Fasudil Hydrochloride (1)

Add 400 ml of methanol to the concentrated solution of compound (6) obtained in Example 3, then add concentrated HCl (36%, 135.5 mL) dropwise, after dripping, raise the temperature to 50-55° C. and stir for 1-2 hours, after detecting complete deprotection through TLC, cool down, and the reaction solution is concentrated under reduced pressure to remove methanol; dilute the resulting concentrated solution with 200 ml of water, adjust the pH≈8, and extract with DCM (200 ml*3); combine the DCM extraction solution and concentrate under reduced pressure to remove DCM to obtain an oily substance; add 400 mL of methanol to dissolve the oily substance, add NaOH (96%, 27.1 g), tetrabutylammonium bromide (99%, 5.3 g), potassium iodide (99%, 2.7 g), reflux for 4 hours, cool down, add 200 ml of water, extract with DCM (300 mL*2), separate the solution, discard the water layer, retain the DCM layer, add 40 g of anhydrous magnesium sulfate, stir and dry, and concentrate under reduced pressure to obtain yellow oily substance; dissolve in 200 ml of ethanol, cool to 0-5° C., adjust the pH of the solution to 5.5-6.0 with concentrated hydrochloric acid, stir for 2 hours, and then concentrate under reduced pressure to obtain crude Fasudil hydrochloride. Recrystallize in a mixed solvent of methanol and isopropyl ether to obtain 83.2 g of white solid Fasudil hydrochloride (1), with an HPLC purity of 99.94% and a total yield of 67.1% (calculated using 5-isoquinoline sulfonyl chloride as the raw material). MP: 247.4-248.2° C. (literature value: 213-215° C.); MS-ESI: 292.1, [M+H]⁺; ¹H NMR (400 MHZ, CDCl₃) δ (ppm): 9.31 (s, 1H), 8.65 (d, J=6.1 Hz, 1H), 8.41 (d, J=6.1 Hz, 1H), 8.31 (dd, J1=7.4 Hz, J2=1.2 Hz, 1H), 8.16 (d, J=8.2 Hz, 1H), 7.66 (t, J=7.7 Hz, 1H), 3.47 (t, J=6.1 Hz, 2H), 3.42 (t, J=5.2 Hz, 2H), 2.95 (t, J=5.4 Hz, 2H), 2.92 (t, J=5.8 Hz, 2H) 1.85-1.78 (m, 2H).

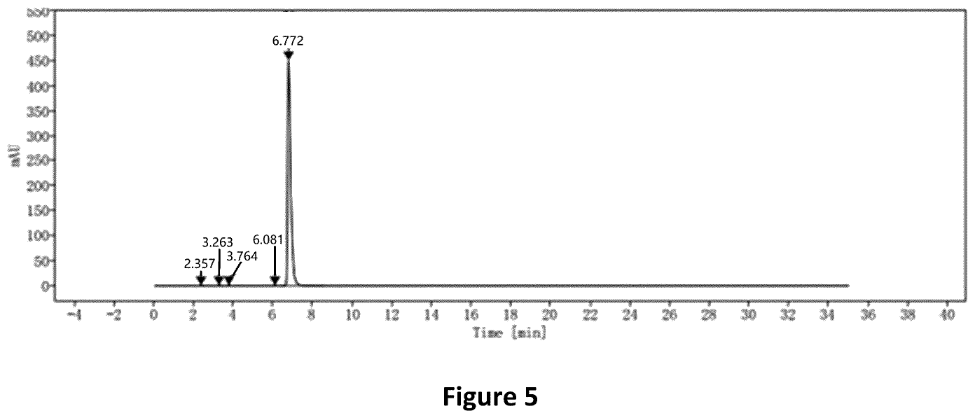

FIG. 5 shows the HPLC of Fasudil Hydrochloride obtained from Example 5, and the relevant information (Detector: UV; Wavelength (nm): 275 nm) is as follows.

| Peak | Retention time | Peak width | Area | Height | |
|---|---|---|---|---|---|
| number | min | min | mV*min | mV | Area % |
| 1 | 2.357 | 0.28 | 0.47 | 0.08 | 0.01 |
| 2 | 3.263 | 0.28 | 1.01 | 0.19 | 0.03 |
| 3 | 3.764 | 0.21 | 0.75 | 0.14 | 0.01 |
| 4 | 6.081 | 0.26 | 0.46 | 0.07 | 0.01 |
| 5 | 6.772 | 2.16 | 5034.37 | 451.05 | 99.94 |
| total | | | 5037.06 | | 100 |

In this specification, the present invention has been described with reference to its specific embodiments. However, it is evident that various modifications and transformations can still be made without departing from the spirit and scope of the present invention. Therefore, the specification is considered explanatory rather than restrictive.

1. A method for preparing compound Fasudil hydrochloride, characterized in that the method comprising:
(1) dissolving 5-isoquinoline sulfonyl chloride in a first organic solvent, then adding dropwise to a mixed solvent of ethylenediamine, solid base, and the first organic solvent, react at 20-25° C. for 2-5 hours, and then subjected to acid-base treatment to obtain N-(2-aminoethyl)-5-isoquinoline sulfonamide;
(2) Mixing the N-(2-aminoethyl)-5-isoquinoline sulfonamide obtained in step (1), base, and a second organic solvent with water, stirring and adding (Boc)₂O solution dropwise, after dropwise addition, reacting for 0.5-2 hours, extracting and separating reaction system after reaction, drying an organic phase and concentrating to obtain tert butyl-(2-(5-isoquinoline sulfonamide)ethyl) carbamate;
(3) Dissolving the tert butyl-(2-(5-isoquinoline sulfonamide)ethyl) carbamate obtained in step (2) in a third organic solvent, and undergoing nucleophilic substitution reaction with 1,3-disubstituted propane at 50-55° C. to obtain compound (6);
(4) Deprotecting compound (6) obtained in step (3) under acidic conditions, then cyclizing under alkaline conditions to obtain fasudil, and then salt with hydrochloric acid to obtain fasudil hydrochloride.

2. The method according to claim 1, characterized in that in step (1), the acid-base treatment is carried out as follows:
Adding hydrochloric acid solution dropwise to reaction solution for adjusting pH to 2-3, and then separating solution, discarding an organic layer;
Dripping an alkaline solution into a water layer for adjusting pH to 7-8, wherein the alkaline solution is sodium bicarbonate, potassium bicarbonate, sodium hydroxide, potassium hydroxide, sodium carbonate or potassium carbonate;
Stirring to precipitate white solid, adding saturated salt water to promote solid precipitation, filtering, and drying filter cake to obtain N-(2-aminoethyl)-5-isoquinoline sulfonamide.

3. The method according to claim 1, characterized in that the first organic solvent is one or more of dichloromethane, dichloroethane, ethylenediamine, triethylamine, diisopropylethylamine, trimethylamine, pyridine, toluene, ethyl acetate, methanol, ethanol, tetrahydrofuran or acetonitrile;
the second organic solvent is one or more of tetrahydrofuran, dioxane, acetonitrile, methanol, or ethanol;
the third organic solvent is one or more of dichloromethane, 1,2-dichloroethane, tetrahydrofuran, dioxane, acetonitrile, methanol, ethanol, ethyl acetate, isopropyl acetate, methyl isobutyl ketone, toluene, isopropyl acetate, N, N-dimethylformamide, N, N-dimethylacetamide, or dimethyl sulfoxide.

4. The method according to claim 1, characterized in that in step (1), the solid base is sodium bicarbonate, potassium bicarbonate, sodium hydroxide, potassium hydroxide, sodium carbonate or potassium carbonate;
in step (2), the base is sodium carbonate, potassium carbonate, sodium bicarbonate, potassium bicarbonate, sodium hydroxide, potassium hydroxide, or triethylamine;
in step (3), the substituent is chlorine, bromine, p-toluenesulfonate, or methanesulfonate;
In step (4), base used for cyclization under alkaline conditions is NaOH, KOH, Na₂CO₃, NaHCO₃, KHCO₃, or triethylamine.

5. The method according to claim 1, characterized in that in step (4), a deprotection condition is a hydrochloric acid/methanol, hydrochloric acid/ethanol, or trifluoroacetic acid/dichloromethane system at a temperature of 50-55° C.
